(19) United States
(12) Patent Application Publication
(10) Pub. No.: US 2017/0202775 A1
(43) Pub. Date: Jul. 20, 2017

(54) METHOD FOR PREPARATION OF LIPID BUBBLES

(71) Applicant: SOUTHEAST UNIVERSITY, Nanjing (CN)

(72) Inventors: Ning GU, Nanjing (CN); Jilai TIAN, Nanjing (CN); Fang YANG, Nanjing (CN); Juan JIN, Nanjing (CN)

(21) Appl. No.: 15/510,644

(22) PCT Filed: Oct. 23, 2015
(86) PCT No.: PCT/CN2015/092758

§ 371 Date: Mar. 10, 2017

(30) Foreign Application Priority Data
Sep. 30, 2014 (CN) 201410522974.6

(51) Int. Cl. A61K 9/127; A61K 47/24; A61K 49/00

(52) U.S. Cl. CPC A61K 9/127; A61K 49/00; A61K 47/24

(57) ABSTRACT

A preparation method of lipid bubbles by utilizing the principle of natural adsorption and assembly of amphiphilic lipid molecules on a gas-liquid interface comprises the steps of mixing gas-dissolved water containing free bubbles with lipid materials, and then the lipid materials are dispersed in the free gas nanobubble aqueous solution, and, in the presence of free bubbles, adsorbed and assembled on the gas-liquid interfaces so as to form lipid bubbles. The method is different from the traditional ultrasound cavitation, the mechanical force action, the novel ink jet printing method, the microchannel method and other methods for preparing bubbles.

This application is the U.S. national phase of International Application No. PCT/CN2015/092758 filed on 23 Oct. 2015 which designated the U.S. and claims priority to Chinese Application Nos. CN201410522974.6 filed on 30 Sep. 2014, the entire contents of each of which are hereby incorporated by reference.

### TECHNICAL FIELD

The invention relates to a preparation method of lipid bubbles, and more particularly to a preparation method of lipid bubbles which can be used in ultrasound contrast and/or drug delivery systems, and belongs to the technical field of medicine.

### BACKGROUND

Large and small bubbles are often seen in nature and life, such as microbubbles in the deep sea, it has an impact on organic particles precipitation rate, ultrasound background scattering and sunlight refraction. Micro-scale bubbles are often used in the fields of ultrasound chemistry, biotechnology and food. In the field of pharmaceutical technology, taking micro-/nano-bubbles as the transport carrier of ultrasound contrast enhancers and/or drugs (including active ingredients such as proteins, genes) is much more mature in today's applications, such as the current commercial microbubble contrast agents Sono Vue, Optison, Levovist, Echogen and so on.

The micro-/nano-bubbles are classified into free gas microbubbles and encapsulated microbubbles in terms of the presence or absence of membrane material. Generally, free gas microbubbles are limited in application due to their short life and rapid gas diffusion rates, while the encapsulated microbubbles generally use membrane material to encapsulate one or some gas of air, sulfur hexafluoride, perfluoropropane, perfluorobutane, and the like, to achieve a more desirable acoustic effect. Phospholipids, albumin, polymers, surfactants and the like are selected as the general membrane material.

The preparation method of encapsulated microbubbles mainly includes ultrasound cavitation, mechanical force action, and new ink jet imprinting method, microfluidic method and the like. The above method is of severe effect and high energy consumption. The invention proposes the preparation of the bubbles by the natural adsorption and assembly of the amphiphilic lipid molecules on the gas-liquid interface. The preparation method has advantages of mild effect, low energy consumption, easy production and use, and convenient promotion.

### SUMMARY

Technical problem: the object of the present invention is to provide a preparation method of lipid bubbles, which is of mild effect and low energy consumption. The prepared lipid microbubbles conform to the ultrasound contrast agent standard.

Technical field: the preparation method of the present invention is that the free gas nanobubble aqueous solution containing free nanobubbles is mixed with lipid material, which is dispersed in the free gas nanobubble aqueous solution and adsorbed and assembled at the gas-liquid interface in the presence of free bubbles, to form lipid bubbles.

The free gas nanobubble aqueous solution is the water, isosmotic solution, isotonic solution or buffer containing nanometer free bubbles.

The gas contained in the free bubbles includes one or more of air, carbon dioxide, oxygen, nitrogen, hydrogen, nitric oxide, hydrogen sulfide, sulfur hexafluoride or perfluoroalkane.

The free gas nanobubble aqueous solution further comprises a pharmaceutically acceptable surfactant.

The pharmaceutically acceptable surfactant comprises Tween, Poloxamer or Phospholipids.

The lipid material comprises a mixture of one or more of egg yolk lecithin, soybean lecithin, hydrogenated egg yolk lecithin, hydrogenated soybean lecithin, sphingomyelin, phosphatidylethanolamine, dimyristoylphosphatidylcholine, dipalmitoylphosphatidylcholine, distearoylphosphatidylcholine, dioleoylphosphatidylcholine, dilaurylphosphatidylcholine, dilaurylphosphatidylglycerol, dimyristoylphosphatidylglycerol, dipalmitoylphosphatidylglycerol, distearoylphosphatidylglycerol, dioleoylphosphatidylglycerol, dilaurylphosphatidic acid, dimyristoylphosphatidic acid, distearoylphosphatidic acid or dioleoylphosphatidylserine.

The lipid material comprises one or more of polyethylene glycol-distearoylphosphatidylethanolamine, polyethylene glycol-polycaprolactone, polyethylene glycol-polyglycolide lactide or polyethylene glycol-polylactic acid.

The lipid material further comprises Tween 80 or Poloxamer 188 as a surfactant.

Beneficial effects: the present invention proposes the preparation of bubbles by natural adsorption and assembly of amphiphilic lipid molecules at the gas-liquid interface, which is different from the conventional ultrasound cavitation and mechanical action including shear emulsification in preparing bubbles. The present invention utilizes the self-assembly principle of the lipid molecule at the gas-liquid interface, proposes to generate free gas nanobubble aqueous solution and then mix it with phospholipid, due to hydrophobic interaction, the freely dispersed phospholipid can be assembled around the free bubbles into the lipid bubbles at the gas-liquid interface. This preparation method is of mild effect and low energy consumption. The prepared lipid microbubbles conform to the ultrasound contrast agent standard.

### BRIEF DESCRIPTION OF THE DRAWINGS

FIG. 1 is the schematic diagram of free gas nanobubble aqueous solution preparation equipment; where A is liquid phase, water here; B is gas phase, sulfur hexafluoride here; C is gas-liquid mixing pump; D is gas-liquid separation device; E is free gas nanobubble aqueous solution tank; F is the prepared sulfur hexafluoride free gas nanobubble aqueous solution;

FIG. 2 shows the Dindal phenomenon of the free gas nanobubble aqueous solution at different time points;

FIG. 3 shows the curve of variation of the surface pressure of different dispersions with different time, where a is ordinary water, b is sulfur hexafluoride free gas nanobubble aqueous solution, c is DDPC added to water, d is DPPC added to sulfur hexafluoride free gas nanobubble aqueous solution.

### DETAILED DESCRIPTION

In the invention, the free gas nanobubble aqueous solution containing free bubbles is mixed with a lipid material, and then the lipid material is dispersed in the gas-dissolved water, and in the presence of free bubbles, adsorbed and assembled at the gas-liquid interface to form lipid bubbles.

The specific method is:

pumping the gas into the water with a gas-liquid mixing pump to produce free gas nanobubble aqueous solution; adding the molted lipid material to the free gas nanobubble aqueous solution and mixing with it to keep the constant pressure stand in the container to form lipid bubbles.

Or pumping the gas into the water or buffer with a gas pump to produce free gas nanobubble aqueous solution; pouring the nanobubble aqueous solution into the container containing lipid material, sealing the container and shaking or stirring to form lipid bubbles.

The free gas nanobubble aqueous solution is the water, isosmotic solution, isotonic solution or buffer containing free bubbles.

The gas contained in the free bubbles includes one or more of air, oxygen, carbon dioxide, nitrogen, hydrogen, nitric oxide, hydrogen sulfide, sulfur hexafluoride or perfluoroalkane.

The free gas nanobubble aqueous solution further comprises a pharmaceutically acceptable surfactant.

The pharmaceutically acceptable surfactant comprises Tween, Poloxamer or Phospholipids.

The lipid material comprises a mixture of one or more of egg yolk lecithin, soybean lecithin, hydrogenated egg yolk lecithin, hydrogenated soybean lecithin, sphingomyelin, phosphatidylethanolamine, dimyristoylphosphatidylcholine, dipalmitoylphosphatidylcholine, distearoylphosphatidylcholine, dioleoylphosphatidylcholine, dilaurylphosphatidylcholine, dilaurylphosphatidylglycerol, dimyristoylphosphatidylglycerol, dipalmitoylphosphatidylglycerol, distearoylphosphatidylglycerol, dioleoylphosphatidylglycerol, dilaurylphosphatidic acid, dimyristoylphosphatidic acid, distearoylphosphatidic acid or dioleoylphosphatidylserine.

The lipid material comprises one or more of polyethylene glycol-distearoylphosphatidylethanolamine, polyethylene glycol-polycaprolactone, polyethylene glycol-polyglycolide lactide or polyethylene glycol-polylactic acid.

The lipid material further comprises Tween 80 or Poloxamer 188 as a surfactant.

### EMBODIMENT 1

### Preparation of Phospholipid Bubbles Containing SF₆

Pumping water with a gas-liquid mixing pump, switching on the SF6 gas delivery valve, adjusting the pressure to 0.3 MPa so as to produce free gas nanobubble aqueous solution containing free bubbles, injecting it into a vial containing 40 mg of phospholipid freeze-dried powder, plugging the lid of the vial immediately and putting the vial aside for 4 hours, and getting phospholipid bubbles containing SF₆.

### EMBODIMENT 2

### Preparation of Phospholipid Bubbles Containing C₃F₈

Pumping Saline solution containing 0.04 g/mL poloxamer 188 with a gas-liquid mixing pump, switching on the C₃F₈ gas delivery valve, adjusting the pressure to 0.3 MPa to produce free gas nanobubble aqueous solution containing free bubbles, injecting it into a vial containing 40 mg of phospholipid freeze-dried powder, plugging the lid of the vial immediately and putting the vial aside for 4 hours, and getting phospholipid bubbles containing C₃F₈.

### EMBODIMENT 3

### Preparation of Phospholipid Bubbles Containing SF₆

Pumping Water is pumped with a gas-liquid mixing pump, switching on the SF₆ gas delivery valve, adjusting the pressure to 0.3 MPa to produce free gas nanobubble aqueous solution containing free bubbles, injecting it into a vial containing 40 mg of phospholipid freeze-dried powder, plugging the lid of the vial immediately, magnetically stirring the vial rapidly for 2 hours, and getting phospholipid bubbles containing SF₆.

### EMBODIMENT 4

### Preparation of Phospholipid Bubbles Containing N₂

Pumping water with a gas-liquid mixing pump, switching on the N₂ gas delivery valve, adjusting the pressure to 0.5 MPa to produce free gas nanobubble aqueous solution containing free bubbles, injecting it into a vial containing 40 mg of egg phospholipid and 2 mg of DSPE-PEG freeze-dried powder, plugging the lid of the vial immediately, putting the vial aside for 2 hours, then subjected to 0.8 μm membrane filtration and getting phospholipid bubbles containing N₂.

### EMBODIMENT 5

### Lipid SF₆ Microbubble Ultrasound Contrast

Pupming water with a gas-liquid mixing pump, switching on the SF₆ gas delivery valve, and free gas nanobubble aqueous solution containing free bubbles is produced and injected into a vial containing phospholipid freeze-dried powder, plugging the lid of the vial immediately, getting phospholipid bubbles containing SF₆ after 0.8 μm membrane filtration. Prepare phantom in terms of a certain proportion of agar powder, glycerine and water, in which the above prepared phospholipid bubbles is injected, the imaging effect is observed at 21 MHz ultrasound frequency. The results show that the imaging effect is obvious, and the imaging time is more than 10 minutes under the ultrasound.

### EMBODIMENT 6

### Preparation and Characterization of Sulfur Hexafluoride Free Gas Nanobubble Aqueous Solution

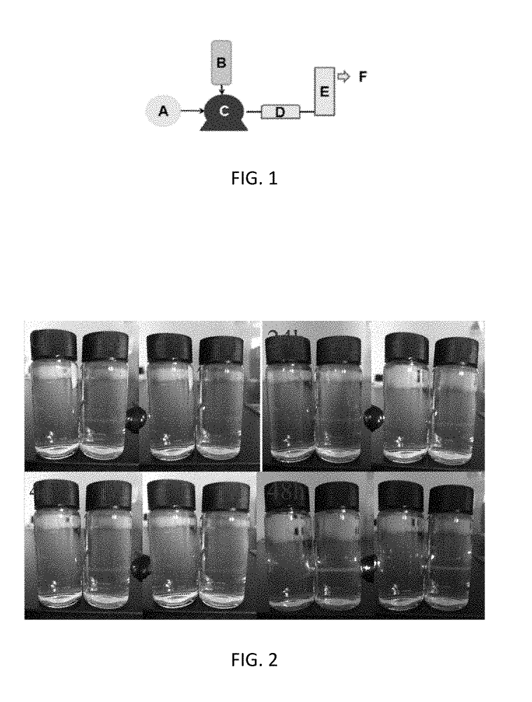

As shown in FIG. 1, setting up the devices, turning on the gas-liquid mixing pump, switching on SF₆ gas delivery valve, adjusting the pressure to 0.3 MPa and keeping the device stable for 20 minutes and collecting free gas nanobubble aqueous solution. The characterizations of free gas nanobubble aqueous solution are as follows:

### (1) Observation of Dindal phenomenon

The Dindal phenomenon of free gas nanobubble aqueous solution is observed and collected at different times, and using ordinary water as a control, as shown in FIG. 2, where the left side of each time point is the control, and the two sets of pictures at each time point represents the left and right directions of the laser light source. 0 point represents the time just to prepare and collect free gas nanobubble aqueous solution.

It can be seen from FIG. 2, compared to ordinary water, the phenomenon of Dindal still exists in the free gas nanobubble aqueous solution after 48 hours, indicating the existence of colloidal particles, while ordinary water does not have this phenomenon. On the one hand, it is proved that the free gas nanobubble aqueous solution is indeed colloid, on the other hand, it is proved the stability of free gas nanobubble aqueous solution.

### (2) Surface Tension Observation

Changes in surface pressure (All) of ordinary water and sulfur hexafluoride free gas nanobubble aqueous solution after DPPC addition can be studied and observed by using Langmuir-Blodgett Troughs (KSV NIMA LB Troughs, Biolin Scientific, Sweden). The surface pressure of ordinary water to air is adjusted to 0, and then the surface pressure change curves of the free gas nanobubble aqueous solution, the ordinary water dripped with DPPC and bubbles water after 48 hours of preparation dripped with DPPC are measured.

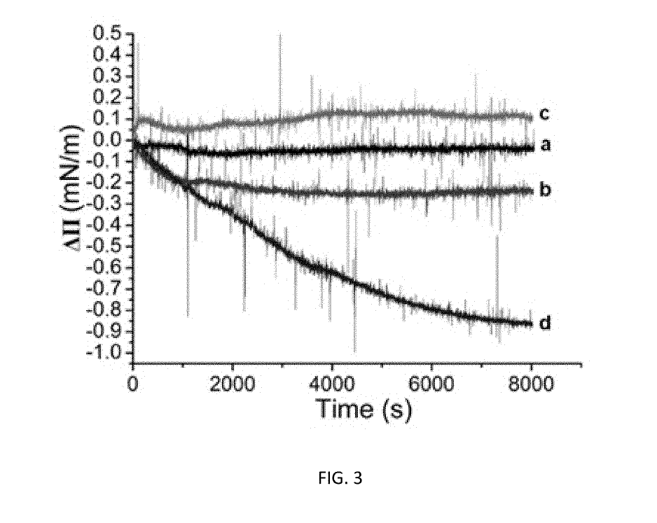

The experimental results are shown in FIG. 3, where a is ordinary water, b is sulfur hexafluoride nanobubble aqueous solution, c is DDPC added to water, d is DPPC added to sulfur hexafluoride nanobubble aqueous solution. The experimental results show that free gas nanobubble aqueous solution has a lower surface tension than ordinary water. DPPC in the free gas nanobubble aqueous solution has increasingly low surface pressure, indicating that the number of the phospholipid molecules at the macro-gas-liquid interface is decreased, which is caused by its entry into the body phase interface.

### EMBODIMENT 7

### Comparison of Particle Sizes of C₃F₈ Phospholipid Bubbles Prepared by Different Preparation Methods

Method one is based on the principle of phospholipid assembly at the free bubble interface, the specific method is to use a pump to pump double distilled water, meanwhile, C₃F₈ gas delivery valve is switched on, and the pressure of 0.3 MPa is adjusted to produce free gas nanobubble aqueous solution containing free bubbles, which is injected into a vial containing 40 mg of phospholipid freeze-dried powder, the lid of the vial is immediately plugged and put the vial aside for 4 hours, so as to prepare phospholipid bubbles containing C₃F₈.

Method two is the traditional shaking method, briefly, the vial containing 40 mg of phospholipid freeze-dried powder (prescription is the same as the method one) is filled with C₃F₈ gas, and the lid of the vial is plugged, the double distilled water is injected and shaken hard for 60 s, so as to prepare phospholipid bubbles containing C₃F₈.

The particle sizes of the phospholipid bubbles prepared by the two methods are determined by particle size analyzer respectively. The results show that the size of particle prepared by method one is (190±12) nm, and the size of particle prepared by method two is (752±240) nm. In the case of polydispersity, the values of lipid bubbles obtained by the method one and method two are 0.2±0.1 and 0.9±0.1, respectively.

### EMBODIMENT 8

### Preparation of Oxygen Nanobubbles Water and Co-Incubation of Oxygen Nanobubbles Water and Molted Phospholipid to Prepare Lipid Bubbles

Preparation method of oxygen nanobubbles water is to use a pump to pump water, switch on the oxygen gas delivery valve, adjust the pressure of 0.3 MPa to produce gas-dissolved water containing nanometer free bubble, which keeps for 20 min to make the system stable, that is to obtain oxygen-loaded free nano-bubbles.

80 mg of egg yolk lecithin and 4 mg of DSPE-PEG are taken and heated to 60° C. to obtain a molted phospholipid. The above prepared oxygen nanobubbles water is injected into the phospholipid material, sealed and standing, then shaken slightly after 4 h and filtered with a 0.8 μm membrane, that is to obtain encapsulated bubbles with phospholipid.

The same method is used to prepare oxygen-free liposomes using ordinary water. The appropriate amount of lipid bubbles and liposomes are sealed in the respective containers, heated to boil, high-precision oxygen analyzer is used to determine the oxygen content of the two. The results show that the oxygen content of oxygen bubble liposomes is 11.3 mg/L, which is higher than that of oxygen-free liposomes, the control group.

### EMBODIMENT 9

### Preparation of Drug-Loaded Lipid Bubbles by Co-Incubation of Sulfur Hexafluoride Nanobubble Water With Coated Phospholipid Containing Homoharringtonine and Their Encapsulation Efficiency Determination

80 mg of egg yolk lecithin, 2 mg of cholesterol, 4 mg of DSPE-PEG and 5 mg of homoharringtonine are taken to be dissolved in absolute ethyl alcohol, and the organic solvent is removed in vacuum to obtain the coated phospholipid. Water is pumped with a gas-liquid mixing pump, SF₆ gas delivery valve is switched on, the pressure of 0.3 MPa is adjusted to produce sulfur hexafluoride dissolved water containing nanometer free bubbles, which keeps stable for 20 min, the prepared free gas nanobubble aqueous solution is injected into the prepared phospholipid material, sealed and standing for 8 h, then shaken slightly and filtered with a 0.8 micron film, that is to obtain drug-loaded lipid bubbles.

The gas-dissolved water in the above method is replaced by degassed water. The preparation method of the degassed water is to boil the double distilled water for half an hour and place it in room temperature. The same method can be used to prepare the drug-loaded phospholipid with degassed water.

Taken the above prepared samples, centrifuged, and the drug content in the supernatant is measured by high performance liquid chromatography, furthermore, the entrapment efficiencies of drug-loaded lipid bubbles and drug-loaded liposome are calculated.

The results show that the encapsulation efficiency of homoharringtonine in drug-loaded liposomes is (74.05±0.54)%, and that is (71.3±6.79)% in group of drug-loaded lipid bubbles, and there is no significant difference between them (P>0.05).

It is confirmed that lipid bubbles prepared by free gas nanobubble water method with a higher drug loading ability.

What is claimed is:

1. A preparation method of lipid bubbles, characterized by the steps of mixing gas nanobubble aqueous solution containing free bubbles with lipid materials, and then the lipid materials are dispersed in the free gas nanobubble aqueous solution, adsorbed and assembled on gas-liquid interfaces when contact to free bubbles so as to form lipid bubbles.

2. The preparation method of lipid bubbles of claim 1, characterized in that the free gas nanobubble aqueous solution refers to the water, isosmotic solution, isotonic solution or buffer containing nanometer scaled free bubbles.

3. The preparation method of lipid bubbles of claim 1, characterized in that the gas contained in the free bubbles includes one or more of air, carbon dioxide, oxygen, nitrogen, hydrogen, nitric oxide, hydrogen sulfide, sulfur hexafluoride or perfluoroalkane.

4. The preparation method of lipid bubbles of claim 1, characterized in that the free gas nanobubble aqueous solution further comprises a pharmaceutically acceptable surfactant.

5. The preparation method of lipid bubbles of claim 4, characterized in that the pharmaceutically acceptable surfactant comprises Tween, Poloxamer or Phospholipids.

6. The preparation method of lipid bubbles of claim 1, characterized in that the lipid materials comprises a mixture of one or more of egg yolk lecithin, soybean lecithin, hydrogenated egg yolk lecithin, hydrogenated soybean lecithin, sphingomyelin, phosphatidylethanolamine, dimyristoylphosphatidylcholine, dipalmitoylphosphatidylcholine, distearoylphosphatidylcholine, dioleoylphosphatidylcholine, dilaurylphosphatidylcholine, dilaurylphosphatidylglycerol, dimyristoylphosphatidylglycerol, dipalmitoylphosphatidylglycerol, distearoylphosphatidylglycerol, dioleoylphosphatidylglycerol, dilaurylphosphatidic acid, dimyristoylphosphatidic acid, distearoylphosphatidic acid or dioleoylphosphatidylserine.

7. The preparation method of lipid bubbles of claim 1, characterized in that the lipid materials comprises one or more of polyethylene glycol-distearoylphosphatidylethanolamine, polyethylene glycol-polycaprolactone, polyethylene glycol-polyglycolide lactide or polyethylene glycol-polylactic acid.

8. The preparation method of lipid bubbles of claim 1, characterized in that the lipid materials further comprises Tween 80 or Poloxamer 188 as a surfactant.
